(19) United States
(12) Patent Application Publication
(10) Pub. No.: US 2016/0263360 A1
(43) Pub. Date: Sep. 15, 2016

(54) UNIVERSAL MULTI-PURPOSE FLUID DRAINAGE CATHETER

(71) Applicant: Reza Mohajer-Shojaee, Encino, CA (US)

(72) Inventor: Reza Mohajer-Shojaee, Encino, CA (US)

(21) Appl. No.: 15/067,974

(22) Filed: Mar. 11, 2016

Related U.S. Application Data
(60) Provisional application No. 62/131,415, filed on Mar. 11, 2015.

(51) Int. Cl. A61M 27/00; A61B 1/06; A61B 1/04; A61M 25/01; A61M 25/00

(52) U.S. Cl. CPC A61M 27/00; A61M 25/01; A61M 25/002; A61B 1/04; A61B 1/0684; A61M 2205/32; A61M 2205/3368

(57) ABSTRACT

A drainage catheter according to aspects of the present invention is generally tubular in shape and has a catheter wall having an external surface and an internal surface, the wall defining an interior lumen of the catheter and an exterior, the wall extending between a proximal end and a distal end wherein the proximal end has a rim and is inserted into a body cavity or false space of a subject when in use for medical treatment and the distal end remains outside the body cavity or false space. The proximal and distal stabilizing elements are integral to the catheter wall and extend outwardly from the catheter wall or are present on opposite ends of a connecting member forming a stabilizer, the stabilizer disposed in the lumen of the catheter, wherein the proximal stabilizing element is positioned distal to the rim of the proximal end of the catheter.

### REFERENCE TO RELATED APPLICATION

This application claims priority from U.S. Provisional Patent Application Ser. No. 62/131,415, filed Mar. 11, 2015, the entire content of which is incorporated herein by reference.

### FIELD OF THE INVENTION

The present invention relates generally to devices and methods for drainage of undesired fluids from a subject's body. According to specific aspects, the present invention relates to drainage devices including a stabilizing element for generally maintaining a position of the device in a subject's body for drainage of undesired fluids therefrom.

### BACKGROUND OF THE INVENTION

For a drainage catheter to work correctly, it is useful to determine that the drainage catheter is properly and securely placed and maintained in a body cavity or open space in the body from which undesired fluid is to be drained.

The Word catheter is currently used to drain fluid from cysts and abscesses in body cavities, especially the Bartholin glands. However, the Word catheters currently being used are inadequate, uncomfortable, painful, require too many peripheral tools, take too many steps to employ, and are costly. The Word catheter balloon expands or has to be manipulated by different tools to complete its function, and may not always stay in place because of the stretched size of the cavity. Furthermore, the Word catheter balloon, in its inflated state, has a potential for blocking the opening of the cavity and drainage of the fluid, counter to its intended purpose of drainage.

Drainage of undesired fluid, such as purulent materials, from body cavities and false spaces continue to be a challenge in surgeries and other medical procedures.

### SUMMARY OF THE INVENTION

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; and a distal stabilizing element extending outwardly from or outwardly through the wall near the distal end.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; a distal stabilizing element extending outwardly from or outwardly through the wall near the distal end; and an introducer, the introducer having an introducer wall, the introducer wall having an external surface and an internal surface, the wall defining an interior lumen of the introducer and an exterior, the wall extending between a proximal end and a distal end of the introducer, wherein the catheter is at least partially disposed in the interior lumen of the introducer.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; a distal stabilizing element extending outwardly from or outwardly through the wall near the distal end; and an introducer, the introducer having an introducer wall, the introducer wall having an external surface and an internal surface, the wall defining an interior lumen of the introducer and an exterior, the wall extending between a proximal end and a distal end of the introducer, wherein the catheter is entirely disposed in the interior lumen of the introducer.

Optionally, at least one handle protrusion is present on the external surface of the introducer at or near the distal end of the introducer.

According to aspects of the present invention, the proximal end of the introducer is a sharp hollow needle-shaped opening.

According to further aspects of the present invention, the proximal end of the introducer is hollow and non-sharp.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; and a distal stabilizing element extending outwardly from or outwardly through the wall near the distal end, wherein the proximal and distal stabilizing elements are integral to the catheter wall and extend outwardly from the catheter wall.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; a distal stabilizing element extending outwardly from or outwardly through the wall near the distal end; and an introducer, the introducer having an introducer wall, the introducer wall having an external surface and an internal surface, the wall defining an interior lumen of the introducer and an exterior, the wall extending between a proximal end and a distal end of the introducer, wherein the catheter is at least partially disposed in the interior lumen of the introducer, wherein the proximal and distal stabilizing elements are integral to the catheter wall and extend outwardly from the catheter wall.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; a distal stabilizing element extending outwardly from or outwardly through the wall near the distal end; and an introducer, the introducer having an introducer wall, the introducer wall having an external surface and an internal surface, the wall defining an interior lumen of the introducer and an exterior, the wall extending between a proximal end and a distal end of the introducer, wherein the catheter is entirely disposed in the interior lumen of the introducer, wherein the proximal and distal stabilizing elements are integral to the catheter wall and extend outwardly from the catheter wall.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; and a distal stabilizing element extending outwardly from or outwardly through the wall near the distal end, wherein the proximal and distal stabilizing elements are present on opposite ends of a connecting member forming a stabilizer, the stabilizer disposed at least partially in the lumen of the catheter, wherein the proximal stabilizing element is positioned distal to the rim of the proximal end of the catheter.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; a distal stabilizing element extending outwardly from or outwardly through the wall near the distal end; and an introducer, the introducer having an introducer wall, the introducer wall having an external surface and an internal surface, the wall defining an interior lumen of the introducer and an exterior, the wall extending between a proximal end and a distal end of the introducer, wherein the catheter is at least partially disposed in the interior lumen of the introducer, wherein the proximal and distal stabilizing elements are present on opposite ends of a connecting member forming a stabilizer, the stabilizer disposed at least partially in the lumen of the catheter, wherein the proximal stabilizing element is positioned distal to the rim of the proximal end of the catheter.

Medical devices are provided according to aspects of the present invention which include a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations; a proximal stabilizing element extending outwardly from or outwardly through the wall near the proximal end; a distal stabilizing element extending outwardly from or outwardly through the wall near the distal end; and an introducer, the introducer having an introducer wall, the introducer wall having an external surface and an internal surface, the wall defining an interior lumen of the introducer and an exterior, the wall extending between a proximal end and a distal end of the introducer, wherein the catheter is entirely disposed in the interior lumen of the introducer, wherein the proximal and distal stabilizing elements are present on opposite ends of a connecting member forming a stabilizer, the stabilizer disposed at least partially in the lumen of the catheter, wherein the proximal stabilizing element is positioned distal to the rim of the proximal end of the catheter.

Optionally, the connector may be attached to the interior surface of the catheter lumen or unattached to the interior surface of the catheter lumen.

A catheter according to aspects of the present invention including a stabilizer has at least one opening in the catheter wall for egress of the proximal stabilizing element.

A catheter according to aspects of the present invention including a stabilizer has at least two proximal stabilizing elements are present and at least two openings in the catheter wall for egress of the at least two proximal stabilizing elements.

Optionally, a catheter according to aspects of the present invention includes a radiopaque x-ray marker disposed on or in the catheter wall.

Optionally, a catheter according to aspects of the present invention includes a fiber optic camera which may be attached to the catheter, preferably at or near the proximal end of the catheter.

Optionally, a catheter according to aspects of the present invention includes a temperature sensor which may be attached to the catheter, preferably attached to the catheter at or near the proximal end of the catheter.

Optionally, a catheter according to aspects of the present invention includes an LED disposed on or in the catheter, such as on the external surface of the catheter wall, the LED in electrical connection with a power source, the power source in operable connection with an activation switch.

Methods of medical treatment of a subject in need thereof are provided according to aspects of the present invention which include administering a catheter of the present invention to the subject, placing the proximal end of the catheter in a body cavity or false space or other location in the subject and the distal end remains outside the body cavity or false space or other location for drainage from the body cavity or false space or other location. Optionally, the catheter is at least partially contained in an introducer during introduction to the subject. Methods of medical treatment of a subject in need thereof are provided according to aspects of the present invention which include administering a drug via the catheter.

The subject has a condition or disease characterized by undesired fluid production or accumulation and wherein treatment of the condition or disease includes removal of a fluid via a catheter according to aspects of the present invention.

### BRIEF DESCRIPTION OF THE DRAWINGS

FIG. 1 shows a universal multi-purpose fluid drainage catheter with location indicator and integral stabilizer at the proximal and distal ends of the catheter;

FIG. 2 shows a universal multi-purpose fluid drainage catheter with location indicator and a stabilizer disposed in the interior of the catheter tubing with stabilizer wings extending from the catheter tubing at or near the proximal and distal ends of the catheter;

FIG. 3 shows an introducer with handles near the distal end of the introducer and a sharp pointed proximal end, containing a universal multi-purpose fluid drainage catheter with location indicator and a stabilizer disposed in the interior of the catheter tubing with stabilizer wings extending from the catheter tubing at or near the proximal and distal ends of the catheter, along with a plunger for urging the drainage catheter out of the introducer into a subject's body;

FIG. 4 shows an introducer with a blunt proximal end, containing a universal multi-purpose fluid drainage catheter with location indicator and a stabilizer disposed in the interior of the catheter tubing with stabilizer wings extending from the catheter tubing at or near the proximal and distal ends of the catheter, along with a plunger for urging the drainage catheter out of the introducer into a subject's body;

FIG. 5 shows a universal multi-purpose fluid drainage catheter with location indicator and a stabilizer disposed in the interior of the catheter tubing;

FIG. 6 shows a universal multi-purpose fluid drainage catheter with location indicator and a stabilizer disposed in the interior of the catheter tubing;

FIG. 7 shows a universal multi-purpose fluid drainage catheter with a stabilizer disposed in the interior of the catheter tubing;

FIG. 8 shows a universal multi-purpose fluid drainage catheter with a stabilizer disposed in the interior of the catheter tubing;

FIG. 9 shows a universal multi-purpose fluid drainage catheter with a stabilizer disposed in the interior of the catheter tubing; and

FIG. 10 shows a universal multi-purpose fluid drainage catheter with a stabilizer disposed in the interior of the catheter tubing.

### DETAILED DESCRIPTION OF THE INVENTION

The singular terms “a,” “an,” and “the” are not intended to be limiting and include plural referents unless explicitly stated otherwise or the context clearly indicates otherwise.

A drainage catheter according to aspects of the present invention is generally tubular in shape and has a catheter wall having an external surface and an internal surface, the wall defining an interior lumen of the catheter and an exterior, the wall extending between a proximal end and a distal end, wherein the proximal end is inserted into a body cavity or false space of a subject when in use for medical treatment and the distal end remains outside the body cavity or false space.

The drainage catheter has one or more perforations through the wall to permit movement of fluids. Where more than one perforation is present in the wall of the drainage catheter, the size and shape of each perforation in the wall may be the same or different than other perforations in the wall of the drainage catheter. Typically the perforations have a longest dimension, such as a diameter, which is less than the internal diameter of the catheter.

The drainage catheter has a proximal stabilizing element for promoting stable retention of the proximal end of the catheter in an area to be drained such as a body cavity or false space in a subject and distal stabilizing element for inhibiting travel of the distal end of the drainage catheter into the area to be drained, thereby promoting drainage of undesirable materials out of the area to be drained.

The proximal and distal stabilizing elements are an integrally formed portion of the catheter or may be reversibly or permanently associated with the catheter according to an aspect of the present invention. A catheter comprising proximal and distal stabilizing elements as an integrally formed portion of the catheter are termed “self-sustaining drainage catheters” herein.

According to aspects of the present invention, the proximal and distal stabilizing elements are present on opposite ends of a connecting member, wherein the combined structure is called a stabilizer herein. A stabilizer is disposed at least partially in the lumen of the catheter such that the proximal and distal stabilizing elements protrude and stabilize the catheter position in use. The proximal stabilizing element protrudes through a slot in the wall of the catheter, the slot extending from the proximal end of the catheter towards the distal end. The slot is generally rectangular and optionally has a generally circular shape at the distal end of the slot. Other slot shapes are possible as long as the proximal stabilizing element is allowed to egress from the slot for anchoring the catheter in use. Where two proximal stabilizing elements are present, two slots are present in the catheter wall. The two slots are preferably generally equidistantly spaced and identical, but they may be different. Where more than two proximal stabilizing elements are present, a slot is present in the catheter wall for egress of each of the proximal stabilizing elements.

The proximal and distal stabilizing elements extend from the proximal and distal ends of the catheter and form an angle with the wall of the catheter allowing the proximal and distal stabilizing elements to inhibit movement of the catheter further into or out of the body cavity or false space into which the proximal end of the catheter is inserted. According to aspects of the present invention, the proximal and distal stabilizing elements extend from the proximal and distal ends of the catheter and form an angle in the range of about 45 to about 135 degrees with the wall of the catheter allowing the proximal and distal stabilizing elements to inhibit movement of the catheter further into or out of the body cavity or false space into which the proximal end of the catheter is inserted.

The proximal and distal stabilizing elements are sized appropriately for stabilizing and maintaining the desired catheter position in the subject. The proximal and distal stabilizing elements can be any shape compatible with their function to stabilize and maintain the position of the catheter. According to particular aspects of the present invention, the proximal and distal stabilizing elements are elongated in shape, having a longest dimension, such as an elongated rectangular or rod-like shape. In general, the proximal and distal stabilizing elements have a longest dimension in the range of 0.25×-5×, 0.3×-4×, 0.4-3×, 0.5-2× or 1-1.5× the longest dimension of a cross section of the catheter, although they can be longer or shorter. For example, where the catheter has a generally circular cross section, the proximal and distal stabilizing elements have a longest dimension in the range of 0.25×-5×, 0.3×-4×, 0.4-3×, 0.5-2× or 1-1.5× the diameter of the cross section of the catheter, although they can be longer or shorter. The proximal and distal stabilizing elements can be the same or different in shape and size. Where multiple proximal stabilizing elements are present, they can be the same or different in shape and size. Similarly, where multiple distal stabilizing elements are present, they can be the same or different in shape and size.

The catheter can be of single piece construction or can be an assembly of components and can be made of any suitable material, typically including flexible materials, including but not limited to, polymers such as but not limited to polypropylene, polyethylene, HDPE, nylon, urethane, polyurethane, PTFE, PEEK and PVDF.

Proximal and distal stabilizing elements are made of flexible materials, including but not limited to, polymers such as but not limited to polypropylene, polyethylene, HDPE, nylon, urethane, polyurethane, PTFE, PEEK and PVDF. The proximal and distal stabilizing elements included in a catheter may be made from the same material as the catheter wall or a different material.

A catheter according to aspects of the present invention is constructed using any suitable manufacturing process, such as, but not limited to, molding, extrusion and combination of these or other manufacturing processes.

A catheter according to aspects of the present invention including integral proximal and distal stabilizing elements is constructed using any suitable manufacturing process, such as, but not limited to, molding, extrusion and combination of these or other manufacturing processes.

A stabilizer is an elongated structure which has a shaft having a proximal end and a distal end with at least one wing extending from the shaft at the proximal end of the shaft and at least one wing extending from the shaft at the distal end of the shaft.

A drainage catheter assembly according to aspects of the present invention includes an introducer for facilitating introduction of a drainage catheter into position in a subject's body for use in medical treatment of the subject. The introducer is generally tubular in shape, having a proximal end adapted for insertion into a subject's body and a distal end adapted for manipulation by a user. The proximal end of the introducer optionally has a cutting edge which is sharp pointed needle-like structure to allow introduction of a drainage catheter into a space or cavity having a wall or other obstruction which must be penetrated to introduce the drainage catheter. The proximal end is optionally open and blunt where cutting penetration is not required.

An introducer has an introducer wall having an external surface and an internal surface, the wall defining an interior lumen of the introducer and an exterior, the wall extending between a proximal end and a distal end wherein the proximal end is inserted into a body cavity or false space of a subject to deliver a drainage catheter at least partially disposed in the interior lumen of the introducer and the distal end remains outside the body cavity or false space and is manipulated by a user introducing the catheter into the subject. Following introduction of the catheter into the subject, the introducer is removed and discarded or sterilized for re-use.

Optionally, the introducer includes one or more handle protrusions at or near the distal end to facilitate handling by a user introducing a drainage catheter into a subject.

The catheter and introducer are suitably sized for introduction into a body cavity or false space as needed for the intended indication. As will be appreciated, the catheter can be used to introduce materials into the body cavity or false space for treating a disease or condition of the subject, such as, but not limited to, a drug. Any drug can be administered via a catheter of the present invention, including, but not limited to, an antibiotic.

A drainage catheter according to aspects of the present invention optionally includes at least one LED location indicator in electrical connection with a power source activated or deactivated by an operable control switch. The power source can be an electrochemical power source, such as a battery or capacitor, or can be an AC or DC power source. In use, a user activates the switch to power the LED location indicator and produce a light. Detection of the light emitted from the LED allows for detection of the location of the drainage catheter in the subject.

A drainage catheter according to aspects of the present invention includes at least one radiopaque X-ray marker disposed on an internal or external surface of the catheter wall, to enable the medical staff to locate and trace the catheter. In a further option, at least one radiopaque X-ray marker is integrated into the catheter wall. For example, the catheter wall may be impregnated with a radiopaque X-ray marker.

A drainage catheter according to aspects of the present invention includes a plunger to urge a drainage catheter into position in a subject.

A drainage catheter according to aspects of the present invention optionally includes a temperature sensor. The temperature sensor is disposed on an internal or external surface of the catheter wall, preferably at or near the proximal end of the catheter. The temperature sensor can be of any type illustratively including a thermocouple, thermistor, temperature sensitive integrated circuit or other temperature sensor. An included temperature sensor transmits a signal via wired or wireless connection to a display for detection of the measured temperature. A temperature sensor can be attached to the drainage catheter during manufacture of the catheter or may be attached by a user when desired. For example, a temperature sensor is optionally reversibly attached to the drainage catheter by a fastener such as a clip.

A drainage catheter according to aspects of the present invention optionally includes a fiber-optic camera. A fiber-optic camera can be attached to the drainage catheter during manufacture of the catheter or may be attached by a user when desired. For example, a fiber-optic camera is optionally reversibly attached to the drainage catheter by a fastener such as a clip. Images from the fiber optic camera may be viewed on a connected display external to the subject's body to examine, for example, structural aspects at the location of the camera and/or progress of a disease or condition of the subject and/or treatment thereof.

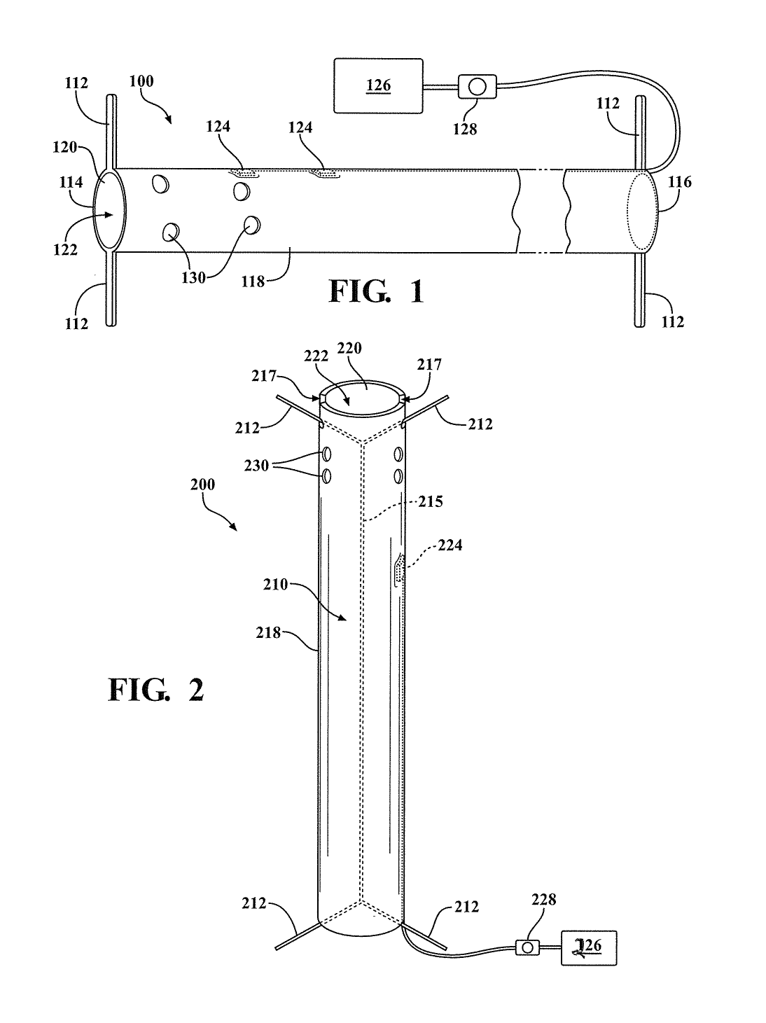

FIG. 1 shows a drainage catheter 100 according to aspects of the present invention with integral stabilizing elements 112 at the proximal 114 and distal 116 ends of the catheter; wherein the catheter is generally tubular in shape and has a catheter wall having an external surface 118 and an internal surface 120, the wall defining an interior lumen 122 of the catheter and an exterior, the wall extending between a proximal end 114 and a distal end 116 wherein the proximal end 114 is inserted into a body cavity or false space of a subject when in use for medical treatment and the distal end 116 remains outside the body cavity or false space. Optional LED locators 124 are shown in electrical connection with a battery power source 126 and operable switch 128. The drainage catheter has one or more perforations 130 through the wall to permit movement of fluids.

FIG. 2 shows a drainage catheter 200 according to aspects of the present invention with a stabilizer 210 having stabilizing elements 212 at the proximal 214 and distal 216 ends of the catheter, the stabilizing elements 212 joined by a connector 215; wherein the catheter is generally tubular in shape and has a catheter wall having an external surface 218 and an internal surface 220, the wall defining an interior lumen 222 of the catheter and an exterior, the wall extending between a proximal end 214 and a distal end 216 wherein the proximal end 214 is inserted into a body cavity or false space of a subject when in use for medical treatment and the distal end 216 remains outside the body cavity or false space. An optional LED locator 224 is shown in electrical connection with a battery power source 226 and operable switch 228. The stabilizing elements 212 are shown in collapsed position and would extend through openings 217 to allow the stabiling elements 212 to attach to tissue. The drainage catheter has one or more perforations 230 through the wall to permit movement of fluids and/or allow stabilizing elements 212 to extend therethrough.

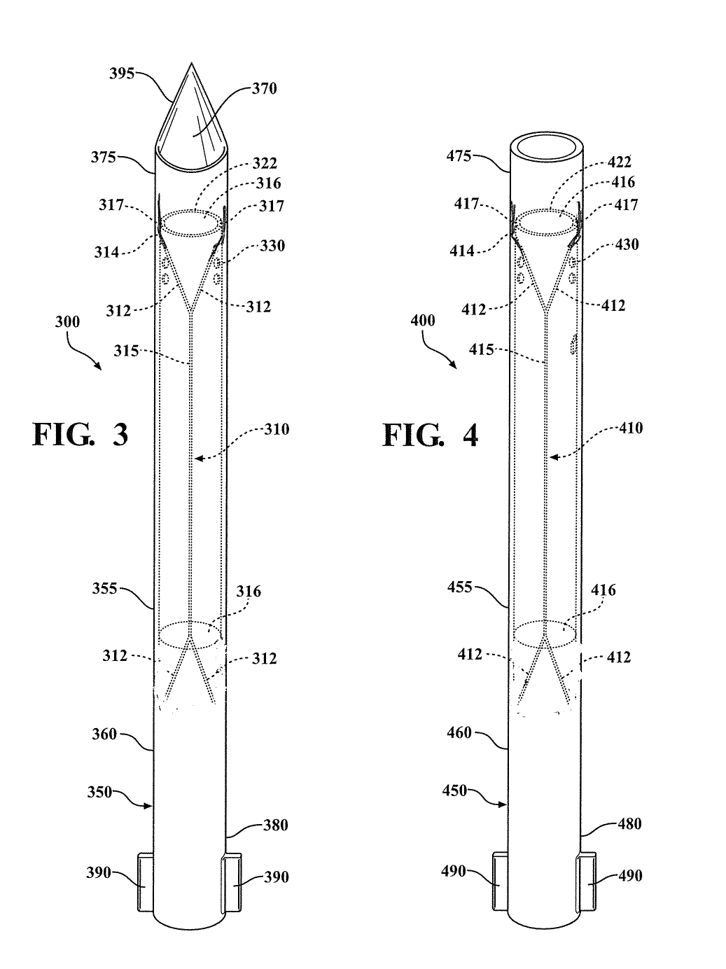

FIG. 3 shows a medical device 300 including a drainage catheter 305 according to aspects of the present invention with a stabilizer 310 having stabilizing elements 312 at the proximal 314 and distal 316 ends of the catheter, the stabilizing elements 312 joined by a connector 315. The drainage catheter has one or more perforations 330 through the wall to permit movement of fluids. The drainage catheter 305 is shown disposed in an introducer 350. The introducer 350 has an introducer wall having an external surface 355 and an internal surface 360, the wall defining an interior lumen 370 of the introducer and an exterior, the wall extending between a proximal end 375 of the introducer and a distal end 380 of the introducer wherein the proximal end 375 is inserted into a body cavity or false space of a subject to deliver a drainage catheter 305 at least partially disposed in the interior lumen 370 of the introducer and the distal end 380 of the introducer remains outside the body cavity or false space and is manipulated by a user introducing the catheter into the subject. The introducer has a sharp needle-like structure 395 to allow introduction of a drainage catheter into a space or cavity having a wall or other obstruction which must be penetrated to introduce the drainage catheter. Optional handle protrusions 390 are shown. The stabilizing elements 312 are shown in collapsed position and would extend through openings 317 to allow the stabiling elements 312 to attach to tissue. The drainage catheter has one or more perforations 330 through the wall to permit movement of fluids and/or allow stabilizing elements 312 to extend therethrough. The stabilizer is positioned below the rim 322 of the catheter. An optional plunger 357 is shown for urging catheter movement through the introducer.

FIG. 4 shows a medical device 400 including a drainage catheter 405 according to aspects of the present invention with a stabilizer 410 having stabilizing elements 412 at the proximal 414 and distal 416 ends of the catheter, the stabilizing elements 412 joined by a connector 415. The drainage catheter 405 is shown disposed in an introducer 450. The introducer 450 has an introducer wall having an external surface 455 and an internal surface 460, the wall defining an interior lumen 470 of the introducer and an exterior, the wall extending between a proximal end 475 of the introducer and a distal end 480 of the introducer wherein the proximal end 475 is inserted into a body cavity or false space of a subject to deliver a drainage catheter 405 at least partially disposed in the interior lumen 470 of the introducer and the distal end 480 of the introducer remains outside the body cavity or false space and is manipulated by a user introducing the catheter into the subject. The introducer has a non-sharp hollow blunt end 495 to allow introduction of a drainage catheter into a space or cavity having a wall or other obstruction which must be penetrated to introduce the drainage catheter. Optional handle protrusions 490 are shown. The stabilizing elements 412 are shown in collapsed position and would extend through openings 417 to allow the stabiling elements 412 to attach to tissue. The drainage catheter has one or more perforations 430 through the wall to permit movement of fluids and/or allow stabilizing elements 412 to extend therethrough. The stabilizer is positioned below the rim 422 of the catheter. An optional plunger 457 is shown for urging catheter movement through the introducer.

According to aspects of the present invention, the introducer is in the form of a hollow tubular needle including appropriate medical-grade metal or a combination of metal and plastic, wherein the tip of the introducer is made of metal and the body of the introducer is made of plastic. The introducer optionally has a sharp tip configured to pierce or lance the body tissues of a subject for the insertion of the drainage catheter into a body cavity. According to aspects of the present invention, the introducer is a tubular metal instrument with a blunt round tip for insertion into an already unobstructed open body cavity.

The introducer is slightly wider in diameter than the drainage catheter and has open distal and proximal ends. The introducer can receive, retain, and push forward the drainage catheter inside its hollow tube, as well as slide outward across the drainage catheter to release and remove it from inside the body cavity or any false space created by trauma or disease. In preferred aspects, the introducer has two handles disposed at opposite sides of the distal end of the introducer.

A drainage catheter according to aspects of the present invention has a distal and a proximal end and is slightly smaller in diameter and shorter than the introducer. The drainage catheter is an open-ended tube including specific polymer blends with particular characteristics or properties to render them suitable and flexible to function as a conduit for the drainage of the body fluids accumulated within the body cavity or false spaces created by trauma and disease.

To avoid slippage and to facilitate drainage, the proximal end of the stabilizer will reside sufficiently below the proximal end of the drainage catheter to keep the drainage catheter securely in-place.

A drainage catheter according to aspects of the present invention has an intact unbroken rim at both the proximal and distal end.

A drainage catheter according to further aspects of the present invention has at its proximal end, two vertical slits located at opposite sides and at equal distance from each other to provide an egress for stabilizing elements of the proximal end of the stabilizer to exit in order to anchor themselves to the adjacent body tissues. A drainage catheter according to further aspects of the present invention has at its proximal end two vertical slits located at opposite sides and at equal distance from each other where each slit terminates in a punched out circular opening through the catheter wall whereby the proximal stabilizing elements of the stabilizer can automatically slip out of the drainage catheter to attach themselves to the adjacent body tissues. The slits are of appropriate size and length to allow egress of the stabilizing elements. Thus, while the drainage catheter provides an egress for the proximal end of the stabilizer, the distal end of the stabilizer remains slightly outside of the catheter's distal end and can be used to pull, dislodge, and remove the drainage catheter out of the body cavity when no longer needed. The proximal end of the drainage catheter extends further than the proximal end of the stabilizer.

The stabilizer structure is configured to include a proximal end and a distal end where one or more stabilizing elements are disposed. Compared to the material of the catheter, the stabilizer includes a stiffer medical-grade polymer material, aiding in preventing the drainage catheter from dislodging and getting lost in the body cavity or false space. The stabilizer further helps the drainage catheter from collapsing inside the body cavity and losing its shape while in-place.

A stabilizer according to aspects of the present invention can be configured and adapted from different shapes that are receptive to being manipulated to perform this anchoring function.

According to aspects of the present invention, an included LED is operated via an alkaline battery, which is less harmful than lithium ion batteries and which does not require any special disposal handling except for the use of the normal medical waste disposal system. The battery is coupled with a manual switch to turn on the LED light when desirous of locating the drain. The manual switch is placed in the portion of the device that remains outside of the body cavity. The LED light will be disposed in the drainage device within the body cavity (or other anatomical areas of the body) requiring drainage and will be encased in a housing of medical grade material. The switch is manipulated by the operator to turn on the LED light to pinpoint the current location of the device. This disclosed LED location indicator can be added to both temporary and permanent medically required drains and catheters used in surgery and in medicine in general.

One or more auxiliary components are hereby disclosed as add-on parts to the drainage catheter of the present invention, as needed. Continuous temperature monitoring is in order for the body cavity when there is fluid collection and buildup, including bleeding and pus. One or both of these parts can be clipped and added onto the drainage catheter at the same time. The advantage of these auxiliary components is close and continuous post-op monitoring of the patient.

A temperature sensor is clipped and added to the tip (proximal end) of the drainage catheter so that when the drainage catheter is inserted into the body cavity of the patient, the patient's body temperature can continuously display on the monitor, reducing the number of times when a patient needs to be physically checked for this purpose.

A fiber-optic camera is clipped and added to the tip of the drainage catheter so that when the drainage catheter is inserted into the body cavity the patient's wide-angled and detailed body cavity information is continuously displayed on the monitor.

Additionally, an optional component of the present invention to be used in some embodiments is a plunger. In some instances, the plunger may be used as a push mechanism in addition to or instead of the introducer to push or slide the drainage catheter forward and backward to facilitate the movement of the drainage catheter inside the body cavity.

Stabilizers can have any of various forms such as, but not limited to, a plurality of petal-like wings structure, a T-shaped structure, an H-shaped structure, a Z-shaped structure, variably joined V-shaped structures, and a numeral 7-shaped structure. Examples of these structures are specifically described and shown herein as to how they are configured and adapted to attach themselves to the adjacent body tissues in order to hold the drainage catheter in-place and to prevent it from losing its location or shape.

According to aspects of the present invention, one piece drainage catheter is provided which is configured to self-contain its stabilizing elements as protruding wings by disposing the anchoring pieces on the proximal end and the distal end of the drainage catheter such that the drainage catheter can be inserted as a self-sustaining unit in the desired position.

According to further aspects, a combination of a drainage catheter and any of the variants of a stabilizer are provided as a preassembled unit for a physician to insert into the introducer for eventual insertion into a body cavity or open spaces of a subject in need thereof. According to further aspects, a combination of a drainage catheter and a stabilizer including V-shaped or inverted V-shaped stabilizing elements are provided as a preassembled unit for a physician to insert into the introducer for eventual insertion into a body cavity or open spaces of a subject in need thereof.

According to aspects of the present invention, the tubular structure of the self-sustained drainage catheter includes a proximal end and a distal end forming a drain lumen therein with perforations in its proximal end. The proximal end splits into a plurality of wing-shaped formations and lie open and expanded in their initial state. The drainage catheter is shorter in length than the introducer to enable it to lodge inside the introducer. The introducer is slightly wider than the drainage catheter in diameter to allow it to receive, retain, and push forward the drainage catheter when inserting the drainage catheter inside the body cavity and to allow it to slide outward across the drainage catheter to remove and release the drainage catheter from within the body cavity.

The sustaining support mechanism (that is, the self-sustained stabilizer) of the tubular structure of the drainage catheter in the body cavity comprises a number of collapsible/expandable wing-shaped structures that are disposed at the proximal and the distal end of the drainage catheter. These wing-shaped structures are separated by openings between and among them, creating multiple pathways, allowing the continuous drainage of the fluid collection. The drainage catheter comprises a blend of suitable polymers combined to attain specific characteristics that would allow these structures to remain open until pushed into a rigid casing such as the slidable metal introducer, at which time they collapse inside the introducer. Upon exit from the introducer, the catheter's wing-shaped structures take their original expanded and opened shape.

A self-sustaining drainage catheter of the present invention, where no separate stabilizer is needed, resides in a collapsed position when inserted into the introducer. The distal end of the self-sustaining drainage catheters will reside inside of the introducer.

In general, the drainage catheter and the other components of the present invention perform the following steps:
1. the self-sustaining one piece unit of the of the drainage catheter and the stabilizer comes ready to be inserted into the introducer;
2. if the drainage catheter is not self-sustaining, a preassembled combination of drainage catheter and the stabilizer comes ready to be inserted into the introducer;
3. a needle-like proximal end of the introducer creates an opening in the body cavity and the false space by lancing or incising the biological site, such as a cyst, abscess, or any other body cavity or false space;
4. a needle-like proximal end of the introducer is removed from the body cavity, leaving behind the drainage catheter inside the body cavity;
5. as the drainage catheter is released from the introducer, its proximal stabilizing elements extend from the catheter, anchoring it to the soft tissues of the body cavity;
6. the wings keep the incised site (or the body cavity) open by preventing the drainage catheter and the cavity from collapsing to allow healing and to prohibit the risk of further infection;
7. the drainage catheter drains the buildup of the body fluids (purulent material, such as blood and pus);
8. the drainage catheter allows introduction of antibiotics into the infected site, should the need arise;
9. the drainage catheter enables irrigation of the site under treatment;
10. the drainage catheter ensures its proper functioning by inhibiting any plugging by tissues and pus;
11. when needed, the manual switch is manipulated to activate the LED light to trace the location of the drain; and
12. Optional add-on pieces. Either a temperature sensor or a fiber-optic camera, if required, can be clipped and added onto the proximal tip of the drainage catheter, separately or simultaneously, to display patient information on the monitoring panel.

Although abscessed cavities, cysts, and false spaces in the body vary in size, because of the materials used and the manufacturing process employed, as soon as the proximal stabilizing elements of the drainage catheter are extended from the catheter inside the cavity, the tissue is gripped by these structures to retain the catheter in place, anchoring themselves to the walls of the body cavity and keeping the site open for as long as the drainage catheter is not intentionally dislodged by the medical staff upon completion of treatment.

In the cases where the body cavity does not need to be incised and either the self-sustained drainage catheter or the combination of the catheter and the stabilizer is used, the introducer with the blunt round proximal end can push the drainage catheter into the body cavity; thus, acting as a plunger. In some cases, a separate push mechanism, such as a generic plunger, can be used to push the drainage catheter into the body cavity instead of the introducer. After the placement of the drainage catheter inside the body cavity, the introducer is withdrawn from the site. The drainage catheter remains in place inside the cavity for the specific number of days prescribed by the surgeon.

When the purulent materials and the fluid collection, such as blood, starts to flow from the drainage catheter it confirms that proper placement of the drainage catheter has taken place. The aggregate plurality of the stabilizing elements and the respective openings between each adjacent structure allows for the flow and complete drainage of the purulent materials in and around the tubular drainage catheter.

When the drainage catheter has to be removed from the healed site or for any other reason, the physician holds the protruding external section and pulls the drainage catheter out. No additional medical tools are required.

According to aspects of the present invention, the tubular structure of the drainage catheter comprises a distal and a proximal end, is slightly smaller in diameter and shorter in length than the introducer and does not have integral stabilizing elements disposed at the proximal end. The stabilizing elements are instead part of a stabilizer of the present invention. The drainage catheter comprises appropriate medical-grade polymer that is blended to particular specifications to enable it to function as a conduit for the drainage of the body fluids.

When a stabilizer is inserted into the drainage catheter for the eventual placement inside the body cavity, the proximal rim of the drainage catheter extends above the proximal end of the stabilizer because the stabilizing elements of the stabilizer will exit from either (1) the equally spaced perforations on either side of the proximal end of the drainage catheter, or (2) from the short slits at each side of the proximal end of the drainage catheter, or (3) from the punched out circular holes that terminate the short slits located at the proximal end of the drainage catheter in any of the variations described herein. In all combined states, the bodies of the drainage catheter and the stabilizer are snug against each other, eliminating any maneuvering room for the separation or dislodgment of the stabilizer from the drainage catheter or the drainage catheter from the body cavity accidentally.

In the following embodiments, where the drainage catheter and the stabilizer are preassembled, the proximal stabilizing elements of the stabilizer are in collapsed position as they lie within the introducer. The distal end of the stabilizer remains outside of the distal end of the drainage catheter and, thus, the introducer. It is further disclosed that the stabilizer's proximal and distal stabilizing elements any of various shapes, such as, but not limited to, those shown herein. For example, a stabilizer may be configured with an uppercase V structure at its proximal end and a reversed uppercase T structure at its distal end.

According to a preferred embodiment of the present invention, a stabilizer comprises two “V-shaped” structures joined together in different configurations and disposed at the opposite ends of a connector that is adapted to coincide with the length of the drainage catheter, as needed. In each case, the stabilizer is slightly narrower in diameter than the drainage catheter tubing and comprises stiffer medical-grade polymer material so as to allow its extensions to retain their shapes both at their collapsed and extended positions. When the drainage catheter and the stabilizer enter the body cavity or open space and reach the desired position, the stabilizing elements of the double-headed V-shaped stabilizer will exit from any one of the egress means e.g. via the perforations, or the slits, or the combination of the slits and the holes, to attach themselves to the body tissue. The catheter's proximal rim resides above the proximal end of the stabilizer with their bodies snug against each other to prohibit any dislodgment of the two components, see for example, FIG. 2. The distal end of the drainage catheter and the distal end of stabilizer will reside outside the body cavity.

According to an aspect of the present invention, two “V-shaped” structures are joined together via a connector emanating from the junction of where the two arms of the distal “V” structure meet and ending at the vertex of the proximal “V” structure, producing a stabilizer. The proximal and the distal stabilizing elements are V-shaped structures where the vertex of each “V” points towards the proximal end of the catheter, where both vertices point towards the distal end of the catheter, where the vertex of the distal V-shaped stabilizing element points towards the distal end of the catheter and the vertex of the proximal V-shaped stabilizing element points towards the proximal end of the catheter or where the vertex of the distal V-shaped stabilizing element points towards the proximal end of the catheter and the vertex of the proximal V-shaped stabilizing element points towards the distal end of the catheter.

According to an aspect of the present invention, the proximal and the distal stabilizing elements are V-shaped structures where the vertex of the distal V-shaped stabilizing element points towards the proximal end of the catheter and the vertex of the proximal V-shaped stabilizing element points towards the distal end of the catheter as shown in FIG. 2.

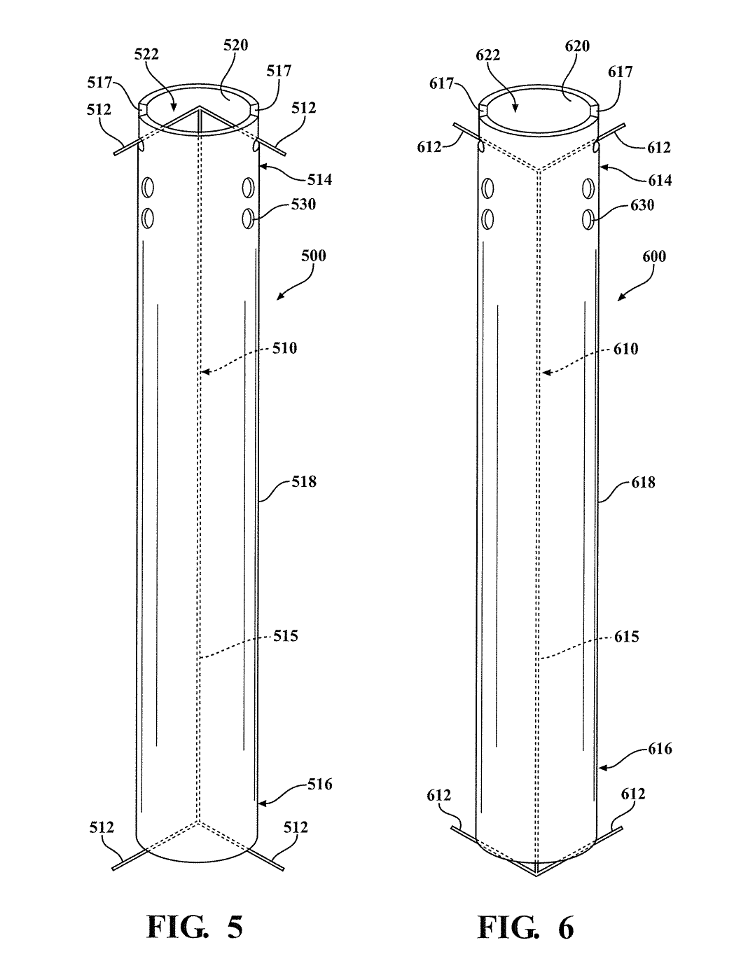

According to an aspect of the present invention, the proximal and the distal stabilizing elements are V-shaped structures which both have vertices that point towards the proximal end of the catheter as shown in FIG. 5. FIG. 5 shows a drainage catheter 500 according to aspects of the present invention with a stabilizer 510 having stabilizing elements 512 at the proximal 514 and distal 516 ends of the catheter, the stabilizing elements 512 joined by a connector 515; wherein the catheter is generally tubular in shape and has a catheter wall having an external surface 518 and an internal surface 520, the wall defining an interior lumen 522 of the catheter and an exterior, the wall extending between a proximal end 514 and a distal end 516 wherein the proximal end 514 is inserted into a body cavity or false space of a subject when in use for medical treatment and the distal end 516 remains outside the body cavity or false space. The stabilizing elements 512 are shown in collapsed position and would extend through openings 517 to allow the stabilizing elements 512 to attach to tissue. The drainage catheter has one or more perforations 530 through the wall to permit movement of fluids and/or allow stabilizing elements 512 to extend therethrough.

According to an aspect of the present invention, the proximal and the distal stabilizing elements are V-shaped structures which both have vertices which point towards the distal end of the catheter as shown in FIG. 6. FIG. 6 shows a drainage catheter 600 according to aspects of the present invention with a stabilizer 610 having stabilizing elements 612 at the proximal 614 and distal 616 ends of the catheter, the stabilizing elements 612 joined by a connector 615; wherein the catheter is generally tubular in shape and has a catheter wall having an external surface 618 and an internal surface 620, the wall defining an interior lumen 622 of the catheter and an exterior, the wall extending between a proximal end 614 and a distal end 616 wherein the proximal end 614 is inserted into a body cavity or false space of a subject when in use for medical treatment and the distal end 616 remains outside the body cavity or false space. The stabilizing elements 612 are shown in collapsed position and would extend through openings 617 to allow the stabilizing elements 612 to attach to tissue. The drainage catheter has one or more perforations 630 through the wall to permit movement of fluids and/or allow stabilizing elements 612 to extend therethrough.

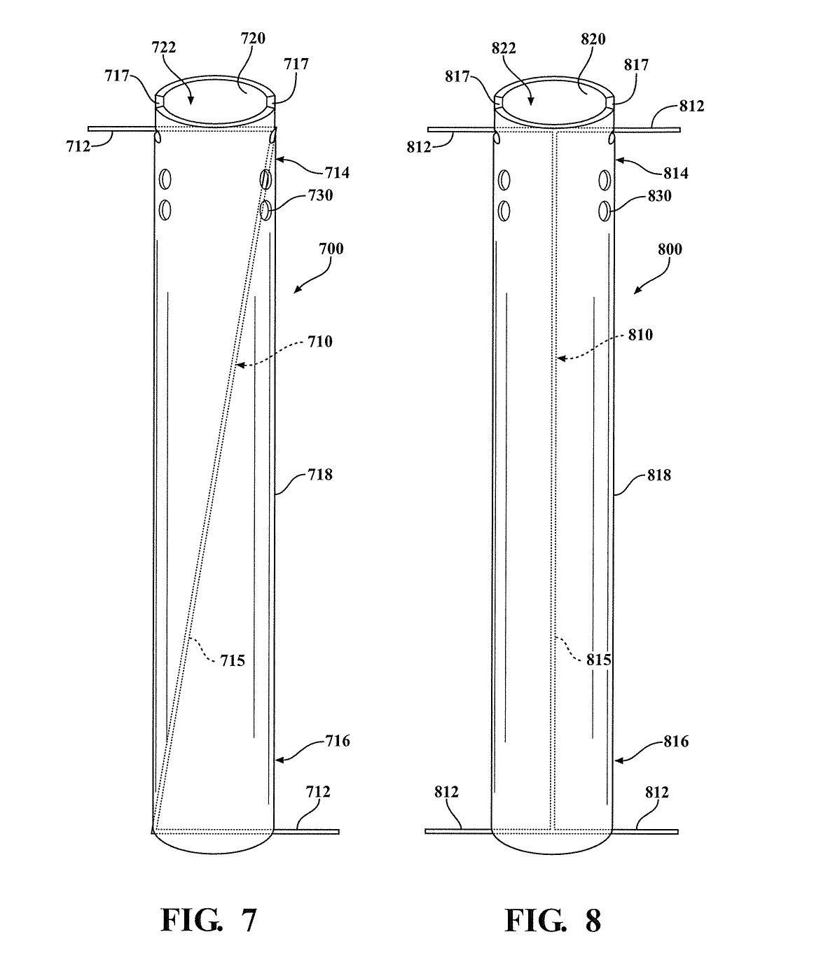

According to an aspect of the present invention, the stabilizer comprises a double-headed “Z-shaped” structure where the horizontal top and bottom stabilizing elements are extended in the opposite directions to achieve the double-headedness (bidirectional) of the stabilizer, as shown in FIG. 7. When the drainage catheter and the stabilizer enter the body cavity or open space and reach the targeted spot, the proximal stabilizing elements of the double-headed Z-shaped stabilizer will exit from any one of the egress means e.g. via the perforations, or the slits, or the combination of the slits and the holes, to attach themselves to the body tissue. In this embodiment, the catheter's proximal rim extends above the proximal end of the stabilizer with their bodies snug against each other to prohibit any dislodgment of the two components, see for example FIG. 2. The distal end of the catheter and the distal end of stabilizer will reside outside the body cavity. FIG. 7 shows a drainage catheter 700 according to aspects of the present invention with a stabilizer 710 having stabilizing elements 712 at the proximal 714 and distal 716 ends of the catheter, the stabilizing elements 712 joined by a connector 715; wherein the catheter is generally tubular in shape and has a catheter wall having an external surface 718 and an internal surface 720, the wall defining an interior lumen 722 of the catheter and an exterior, the wall extending between a proximal end 714 and a distal end 716 wherein the proximal end 714 is inserted into a body cavity or false space of a subject when in use for medical treatment and the distal end 716 remains outside the body cavity or false space. The stabilizing elements 712 are shown in collapsed position and would extend through openings 717 to allow the stabilizing elements 712 to attach to tissue. The drainage catheter has one or more perforations 730 through the wall to permit movement of fluids and/or allow stabilizing elements 712 to extend therethrough.

According to an aspect of the present invention, the drainage catheter includes a stabilizer wherein a double-headed uppercase “T-shaped” structure's descender is extended in both directions to create the double-headedness (bidirectional flaps) of the shape as shown in FIG. 8 to function as the stabilizer. In this embodiment, the stabilizer comprises a central connector with long proximal and distal stabilizing elements extending horizontally from opposite sides of the proximal and distal ends of the connector. The stabilizer is configured appropriately from medical-grade polymer material (stiffer and slightly narrower in diameter than the catheter material) so as to ensure it retains its shape. When inserted into the drainage catheter, the stabilizing elements of the stabilizer will be in their collapsed position. Once the preassembled combination of the catheter and stabilizer are pushed, via the introducer, into the body cavity or open space, the long stabilizing elements open up horizontally and attach themselves to the adjacent body tissues. In these embodiments, the catheter's proximal rim extends above the proximal end of all the stabilizers. The stabilizing elements of the double-headed T-shaped stabilizer will exit from any one of the egress means e.g., via the perforations, or the slits, or the combination of the slits and the holes. The bodies of the drainage catheter and the stabilizer will be snug against each other, prohibiting any dislodgment of the two components, see for example, FIG. 2. The distal end of the drainage catheter and the stabilizer will remain outside the body cavity. FIG. 8 shows a drainage catheter 800 according to aspects of the present invention with a stabilizer 810 having stabilizing elements 812 at the proximal 814 and distal 816 ends of the catheter, the stabilizing elements 812 joined by a connector 815; wherein the catheter is generally tubular in shape and has a catheter wall having an external surface 818 and an internal surface 820, the wall defining an interior lumen 822 of the catheter and an exterior, the wall extending between a proximal end 514 and a distal end 816 wherein the proximal end 814 is inserted into a body cavity or false space of a subject when in use for medical treatment and the distal end 816 remains outside the body cavity or false space. The stabilizing elements 812 are shown in collapsed position and would extend through openings 817 to allow the stabilizing elements 812 to attach to tissue. The drainage catheter has one or more perforations 830 through the wall to permit movement of fluids and/or allow stabilizing elements 812 to extend therethrough.

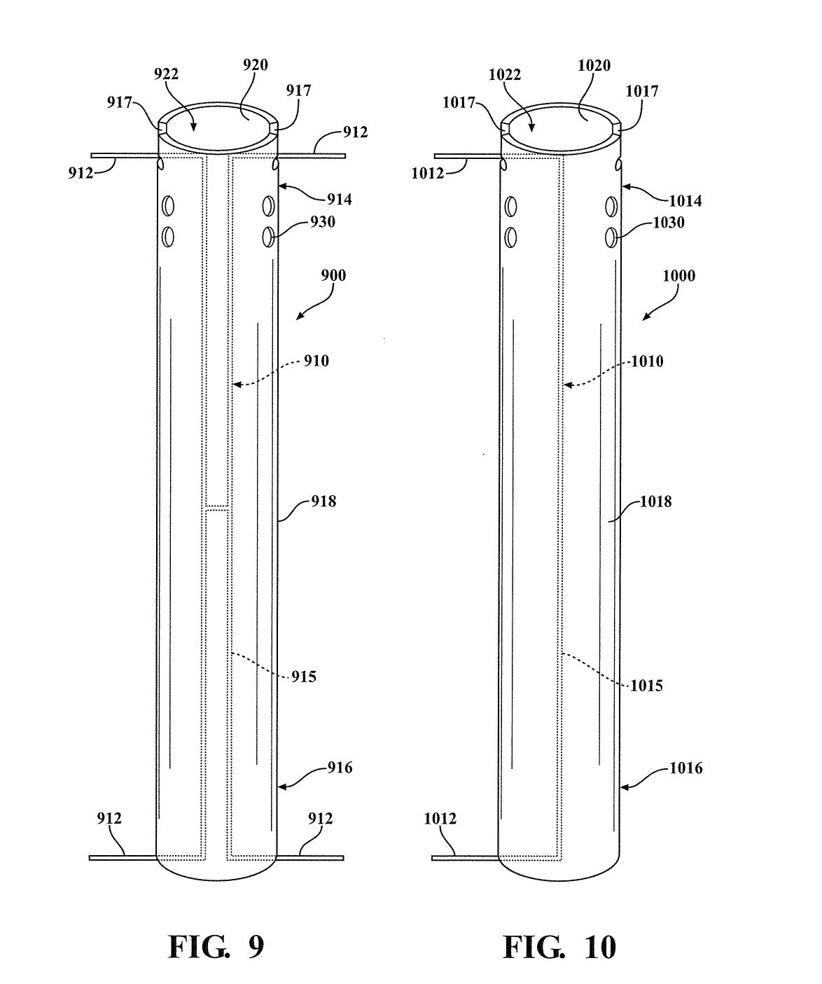

According to an aspect of the present invention, the stabilizer comprises a double-headed uppercase “H-shaped” structure with horizontally extended stabilizing elements to form a double-headed anchor whose sides are connected at midpoint via a bar, as shown in FIG. 9. The stabilizer structure includes four wing-like extensions, one at each terminating end of the stems of the “H” structure. The structure is slightly narrower in diameter than the drainage catheter tubing and comprises stiffer medical-grade polymer material so as to allow its extensions to retain their shapes both at their collapsed and extended positions. When the stabilizer enters the drainage catheter, its proximal stabilizing elements will exit from any one of the egress means e.g. via the perforations, or the slits, or the combination of the slits and the holes to anchor themselves to the body tissue. The bodies of the drainage catheter and the stabilizer will be snug against each other, prohibiting any dislodgment of the two components, see for example, FIG. 2. The distal end of the drainage catheter and the stabilizer will remain outside the body cavity. FIG. 9 shows a drainage catheter 900 according to aspects of the present invention with a stabilizer 910 having stabilizing elements 912 at the proximal 914 and distal 916 ends of the catheter, the stabilizing elements 912 joined by a connector 915; wherein the catheter is generally tubular in shape and has a catheter wall having an external surface 918 and an internal surface 920, the wall defining an interior lumen 922 of the catheter and an exterior, the wall extending between a proximal end 914 and a distal end 916 wherein the proximal end 914 is inserted into a body cavity or false space of a subject when in use for medical treatment and the distal end 916 remains outside the body cavity or false space. The stabilizing elements 912 are shown in collapsed position and would extend through openings 917 to allow the stabilizing elements 912 to attach to tissue. The drainage catheter has one or more perforations 930 through the wall to permit movement of fluids and/or allow stabilizing elements 912 to extend therethrough.

FIG. 10 shows a drainage catheter 1000 according to aspects of the present invention with a stabilizer 910 having stabilizing elements 1012 at the proximal 1014 and distal 1016 ends of the catheter, the stabilizing elements 1012 joined by a connector 1015; wherein the catheter is generally tubular in shape and has a catheter wall having an external surface 918 and an internal surface 1020, the wall defining an interior lumen 1022 of the catheter and an exterior, the wall extending between a proximal end 1014 and a distal end 1016 wherein the proximal end 1014 is inserted into a body cavity or false space of a subject when in use for medical treatment and the distal end 1016 remains outside the body cavity or false space. The stabilizing elements 1012 are shown in collapsed position and would extend through openings 1017 to allow the stabilizing elements 1012 to attach to tissue. The drainage catheter has one or more perforations 1030 through the wall to permit movement of fluids and/or allow stabilizing elements 1012 to extend therethrough.

According to an aspect of the present invention, the stabilizer comprises two uppercase “L-shaped” structures whose stems are joined back-to-back to create a double-headed reversed uppercase L structure. The ascenders of the combined structures are extended in the opposite directions of the existing proximal and distal stabilizing elements to achieve its bidirectional left and right flaps (double-headedness). The top and bottom projections stabilizing elements are in collapsed position as they lie within the drainage catheter. When the drainage catheter and the stabilizer enter the body cavity or open space and reach the targeted spot, the proximal stabilizing elements of the double-headed L-shaped stabilizer will exit from any one of the egress means e.g., via the perforations, or the slits, or the combination of the slits and the holes to attach themselves to the body tissue. In this embodiment, the catheter's proximal rim resides above the proximal end of the stabilizer with their bodies snug against each other to prohibit any dislodgment of the two components, see for example FIG. 2. The distal end of the drainage catheter and the distal end of stabilizer will reside outside the body cavity.

According to an aspect of the present invention, the stabilizer comprises a double-headed “numeral 7” where the two “7” numerals are joined in reverse vertical direction back-to-back with the descenders extended at opposite directions of the existing flaps to create the bidirectional or the double-headed numeral “7”. The proximal stabilizing elements of the stabilizer are in collapsed position as they lie within the introducer. The distal end of the stabilizer remains outside of the distal end of the drainage catheter and, thus, the introducer. When the drainage catheter and the stabilizer enter the body cavity or open space and reach the targeted spot, the proximal stabilizing elements of the 7 numeral stabilizer will exit from any one of the egress means e.g. via the perforations, or the slits, or the combination of the slits and the holes to attach themselves to the body tissue. In this embodiment, the catheter's proximal rim extends above the proximal end of the stabilizer with their bodies snug against each other to prohibit any dislodgment of the two components for example as shown in FIG. 2. The distal end of the drainage catheter and the distal end of stabilizer will reside outside the body cavity.

Methods of medical treatment of a subject in need thereof are provided according to aspects of the present invention which include administering a catheter having a catheter wall, the catheter wall having an external surface and an internal surface, the wall defining an interior lumen of the catheter and an exterior, the wall extending between a proximal end and a distal end and the wall having one or more perforations; a proximal stabilizing element extending outwardly from or outwardly through the wall near the proximal end; and a distal stabilizing element extending outwardly from or outwardly through the wall near the distal end, to the subject. According to aspects of the present invention, the catheter is at least partially contained in an introducer.

A subject treated using a catheter according to aspects of the present invention can be an individual of any age, either sex and of any of various species. While the present invention describes medical devices and methods for treatment of human subjects in need thereof, the present invention is not limited to human subjects and the term subject generally includes mammals and birds, such as, but not limited to, non-human primates, cats, dogs, cows, horses, rodents, pigs, sheep, goats and poultry. Subjects are identified as in need of medical treatment including administration of a catheter using well-known medical and diagnostic techniques. The terms “treating” and “treatment” and grammatical equivalents thereof used to refer to treatment of a condition or disease in a subject include: inhibiting or ameliorating the condition or disease in the subject.

A subject in need of treatment according to aspects of the present invention has a condition or disease characterized by undesired fluid production or accumulation and wherein treatment of the condition or disease includes removal of the fluid. Examples of fluids removed according to aspects of the present invention include, but are not limited to, blood, pus, urine, wound exudate and ascites.

A drug may be administered to the subject via a catheter having a catheter wall, the catheter wall having an external surface and an internal surface, the wall defining an interior lumen of the catheter and an exterior, the wall extending between a proximal end and a distal end and the wall having one or more perforations; a proximal stabilizing element extending outwardly from or outwardly through the wall near the proximal end; and a distal stabilizing element extending outwardly from or outwardly through the wall near the distal end according to aspects of the present invention.

An administered catheter is at least partially contained in an introducer according to aspects of the present invention.

The term “subject” refers to an individual in need of treatment for a pathological condition responsive to the beneficial effects of compositions of the present invention, particularly cancer. While the present invention describes compositions and methods for treatment of human subjects in need thereof, the present invention is not limited to human subjects and the term subject generally includes mammals and birds, such as, but not limited to, non-human primates, cats, dogs, cows, horses, rodents, pigs, sheep, goats and poultry.

Commercial Packages

Commercial packages are provided according to aspects of the present invention including a catheter having a catheter wall, the catheter wall having an external surface and an internal surface, the wall defining an interior lumen of the catheter and an exterior, the wall extending between a proximal end and a distal end and the wall having one or more perforations; a proximal stabilizing element extending outwardly from or outwardly through the wall near the proximal end; and a distal stabilizing element extending outwardly from or outwardly through the wall near the distal end. One or more auxiliary components are optionally included in commercial packages of the present invention, including, but not limited to, an LED position locator, a fiber optic camera and a temperature sensor. One or more fasteners or adhesives may be included for fastening an auxiliary component to the catheter. Alternatively, the catheter may already include an auxiliary component fastened thereto. Instructions for use of the catheter are optionally included in a commercial package according to aspects of the present invention.

An introducer is optionally included in a commercial package according to aspects of the present invention. A catheter may be disposed at least partially inside the introducer in the commercial package or these components may be included separately for assembly by a user.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A medical device, comprising:
a catheter having a catheter wall, the catheter wall having an external surface and an internal surface, the wall defining an interior lumen of the catheter and an exterior, the wall extending between a proximal end and a distal end, the proximal end having a rim, and the wall having one or more perforations;
a proximal stabilizing element extending outwardly from or outwardly through the wall near the proximal end; and
a distal stabilizing element extending outwardly from or outwardly through the wall near the distal end.

2. The medical device of claim 1, further comprising an introducer, the introducer having an introducer wall, the introducer wall having an external surface and an internal surface, the wall defining an interior lumen of the introducer and an exterior, the wall extending between a proximal end and a distal end of the introducer, wherein the catheter is at least partially disposed in the interior lumen of the introducer.

3. The medical device of claim 1, wherein at least one handle protrusion is present on the external surface of the introducer at or near the distal end of the introducer.

4. The medical device of claim 2, wherein the proximal end of the introducer is a sharp hollow needle-shaped opening.

5. The medical device of claim 2, wherein the proximal end of the introducer is hollow and non-sharp.

6. The medical device of claim 1, wherein the proximal and distal stabilizing elements are integral to the catheter wall and extend outwardly from the catheter wall.

7. The medical device of claim 1, wherein the proximal and distal stabilizing elements are present on opposite ends of a connecting member forming a stabilizer, the stabilizer disposed at least partially in the lumen of the catheter, wherein the proximal stabilizing element is positioned distal to the rim of the proximal end of the catheter.

8. The medical device of claim 7, wherein the connector is attached to the interior surface of the catheter lumen.

9. The medical device of claim 7, wherein the connector is not attached to the interior surface of the catheter lumen.

10. The medical device of claim 7, further comprising at least one opening in the catheter wall for egress of the proximal stabilizing element.

11. The medical device of claim 10, wherein at least two proximal stabilizing elements are present and at least two openings in the catheter wall for egress of the at least two proximal stabilizing elements.

12. The medical device of claim 1, further comprising a radiopaque x-ray marker disposed on or in the catheter wall.

13. The medical device of claim 1, further comprising a fiber optic camera attached to the catheter at or near the proximal end of the catheter.

14. The medical device of claim 1, further comprising a temperature sensor attached to the catheter at or near the proximal end of the catheter.

15. The medical device of claim 1, further comprising an LED disposed on the external surface of the catheter wall, the LED in electrical connection with a power source, the power source in operable connection with an activation switch.

16. A method of medical treatment of a subject in need thereof, comprising:
administering a catheter according to claim 1 to the subject.

17. The method of claim 16, wherein the catheter is at least partially contained in an introducer.

18. The method of claim 16, wherein the subject has a condition or disease characterized by undesired fluid production or accumulation and wherein treatment of the condition or disease comprises removal of the fluid via the catheter according to claim 1.

19. The method of claim 16, further comprising administration of a drug via the catheter according to claim 1.

20. A commercial package comprising a catheter according to claim 1.
